(19) United States
(12) Patent Application Publication
(10) Pub. No.: US 2025/0250313 A1
(43) Pub. Date: Aug. 7, 2025

(54) IL-15 AGONISTS FOR CANCER

(71) Applicant: REMEGEN CO., LTD., Yantai (CN)

(72) Inventors: Zhulun WANG, South San Francisco, CA (US); Xiaoshan MIN, South San Francisco, CA (US); Hang CHEN, South San Francisco, CA (US)

(21) Appl. No.: 18/855,646

(22) PCT Filed: Feb. 19, 2024
(86) PCT No.: PCT/CN2024/077535

§ 371 Date: Oct. 10, 2024

Related U.S. Application Data
(60) Provisional application No. 63/486,185, filed on Feb. 21, 2023.

(51) Int. Cl. C07K 14/54; A61K 38/00; A61P 35/04; C07K 14/715

(52) U.S. Cl. CPC C07K 14/5443; A61P 35/04; C07K 14/7155; A61K 38/00; C07K 2319/30

(57) ABSTRACT

Provided herein are IL-15/IL-15Rα-Fc fusion proteins that comprise an IL-15 receptor alpha sushi domain, an IL-15 C-terminal domain, an IL-15 N-terminal domain; and an Fc domain. Further provided are methods of using such IL-15/IL-15Rα-Fc to treat diseases and disorders, such as cancer.

### FIELD OF THE DISCLOSURE

The present disclosure relates to interleukin-15 (IL-15) agonists and methods of use thereof. Specifically, the present disclosure provides an IL-15/IL-15 receptor alpha (IL-15Rα)-Fc fusion protein complex including an IL-15Rα sushi domain, an IL-15 domain, and an Fc domain for the treatment of cancer.

### BACKGROUND

Cancer immunotherapy represents a promising avenue for cancer treatment, but is currently limited due to poor drug-like properties and severe dose-limiting toxicities, as well as disease recurrence. Thus, there remains a need for effective compositions that augment and direct immune activity against cancer cells with limited toxicity. The present disclosure provides novel IL-15/IL-15Rα-Fc fusion proteins that activate effector immune responses for the treatment of cancer.

### BRIEF SUMMARY

The present disclosure provides an IL-15/IL-15Rα-Fc fusion protein including from N-terminus to C-terminus: (a) an IL-15 receptor alpha sushi domain; (b) an IL-15 C-terminal domain; (c) an IL-15 N-terminal domain; and (d) an Fc domain.

In some embodiments, the IL-15 receptor alpha sushi domain includes an amino acid sequence with at least about 85%, at least about 90%, at least about 95%, at least about 96%, at least about 97%, at least about 98%, or at least about 99% sequence identity to any one of SEQ ID NOs: 8-11. In some embodiments, the IL-15 receptor alpha sushi domain includes an amino acid sequence of any one of SEQ ID NOs: 8-11.

In some embodiments, the IL-15 C-terminal domain includes an amino acid sequence with at least about 85%, at least about 90%, at least about 95%, at least about 96%, at least about 97%, or at least about 98% sequence identity to SEQ ID NO: 3 or SEQ ID NO: 4. In some embodiments, the IL-15 C-terminal domain includes an amino acid sequence of SEQ ID NO: 3 or SEQ ID NO: 4.

In some embodiments, the IL-15 N-terminal domain includes an amino acid sequence with at least about 85%, at least about 90%, at least about 95%, at least about 96%, at least about 97%, or at least about 98% sequence identity to SEQ ID NO: 5 or SEQ ID NO: 6. In some embodiments, the IL-15 N-terminal domain includes an amino acid sequence of SEQ ID NO: 5 or SEQ ID NO: 6. In some embodiments, the IL-15 N-terminal domain includes an N72D mutation.

In some embodiments, the Fc domain is an IgG1 Fc domain including an amino acid sequence with at least about 85%, at least about 90%, at least about 95%, at least about 96%, at least about 97%, at least about 98%, or at least about 99% sequence identity to SEQ ID NO: 12. In some embodiments, the Fc domain is an IgG1 Fc domain including an amino acid sequence of SEQ ID NO: 12.

In some embodiments, the Fc domain is an IgG4 Fc domain including an amino acid sequence with at least about 85%, at least about 90%, at least about 95%, at least about 96%, at least about 97%, at least about 98%, or at least about 99% sequence identity to SEQ ID NO: 17. In some embodiments, the Fc domain is an IgG4 Fc domain including an amino acid sequence of SEQ ID NO: 17.

In some embodiments, the IgG1 or IgG4 Fc domain includes at least one amino acid substitution. In some embodiments, the at least one amino acid substitution is N297A (EU numbering scheme). In some embodiments, the at least one amino acid substitution is L234A, L235A, and/or P329G (EU numbering scheme). In some embodiments, the at least one amino acid substitution is M252Y, S254T, T256E (EU numbering scheme), or a combination thereof. In some embodiments, the at least one amino acid substitution is L234A, L235A, or a combination thereof.

In some embodiments, the IL-15/IL-15Rα-Fc fusion protein includes one or more linkers. In some embodiments, the IL-15/IL-15Rα-Fc fusion protein includes a linker between the IL-15 C-terminal domain and the IL-15 N-terminal domain. In some embodiments, the IL-15/IL-15Rα-Fc fusion protein includes a linker between the IL-15 N-terminal domain and the Fc domain. In some embodiments, the IL-15/IL-15Rα-Fc fusion protein includes a linker between the IL-15 C-terminal domain and the IL-15 N-terminal domain; and a linker between the IL-15 N-terminal domain and the Fc domain. In some embodiments, the one or more linkers is 0, 2, 3, 4, 5, 6, 7, 8, 9, or 10 amino acids in length. In some embodiments, the one or more linkers is 0 or 5 amino acids in length. In some embodiments, the one or more linkers include an amino acid sequence selected from the group consisting of: GG, GS, GGS, GGGS, and GGGGS.

In some embodiments, the IL-15/IL-15Rα-Fc fusion protein includes an amino acid sequence with at least about 85%, at least about 90%, at least about 95%, at least about 96%, at least about 97%, at least about 98%, or at least about 99% sequence identity to any one of SEQ ID NOs: 20-30. In some embodiments, the IL-15/IL-15Rα-Fc fusion protein includes an amino acid sequence of any one of SEQ ID NOs: 20-30. In some embodiments, the IL-15/IL-15Rα-Fc fusion protein consists of an amino acid sequence of any one of SEQ ID NOs: 20-30.

In some embodiments, the IL-15/IL-15Rα-Fc fusion protein includes an amino acid sequence of any one of SEQ ID NOs: 20-30 with one or more mutations. In some embodiments, the IL-15/IL-15Rα-Fc fusion protein includes an amino acid sequence of SEQ ID NO: 24 with one or more mutations selected from the group consisting of: V3A, V3L, N4A, N4D, N4Q, S7D, S7V, D8T, D8A, K11A, D30N, D61A, D61S, D61N, D61E, E64Q, N65D, 168A, 168V, L69A, L69V, N72E, and N72A (The above-reference amino acid positions correspond to the natural ordering positions of amino acids in SEQ ID NO: 5).

In some embodiments, the IL-15/IL-15Rα-Fc fusion protein includes an amino acid sequence of SEQ ID NO: 28 with one or more mutations selected from the group consisting of: N189Q (referring to the mutation at N (Asparagine) 189 to G (Glycine) in SEQ ID NO: 28) and G190A (referring to the mutation at G (Glycine) 190 to A (Alanine) in SEQ ID NO: 28). In some embodiments, the IL-15/IL-15Rα-Fc fusion protein includes an amino acid sequence of SEQ ID NO: 30 with one or more mutations selected from the group consisting of: N189Q and G190A.

In some embodiments, the IL-15/IL-15Rα-Fc fusion protein binds to IL-2 receptor beta and/or IL-2 receptor gamma. In some embodiments, the IL-15/IL-15Rα-Fc fusion protein binds to IL-2 receptor beta with an affinity of about 0.1 nM to about 25 nM, about 0.5 nM to about 10 nM, or about 1 nM to about 5 nM.

In some embodiments, the present disclosure provides a polynucleotide encoding the IL-15/IL-15Rα-Fc fusion protein described above. In some embodiments, the present disclosure provides an expression vector including the polynucleotide. In some embodiments, the present disclosure provides a host cell including the polynucleotide or the expression vector.

In some embodiments, the present disclosure provides a pharmaceutical composition including the IL-15/IL-15Rα-Fc fusion protein described above, and at least one pharmaceutically acceptable carrier or excipient.

In some embodiments, the present disclosure provides a method of treating cancer, the method including administering to a subject in need thereof an effective amount of the IL-15/IL-15Rα-Fc fusion protein described herein, or a pharmaceutical composition thereof.

In some embodiments, the cancer is a solid tumor. In some embodiments, the cancer is metastatic. In some embodiments, the cancer is relapsed or refractory to treatment.

### BRIEF DESCRIPTION OF THE DRAWINGS

FIG. 1A-FIG. 1C show an exemplary 3D x-ray diffraction (XRD) structure of the IL-15 complex (PDB: 4GS7). FIG. 1A and FIG. 1B show combined ribbon structure and surface view of an IL-15 quaternary complex from different perspectives. FIG. 1C shows the interface between IL-15 and IL-15Rα within the quaternary complex. Dashed lines denote where linkers were placed to join the IL-15 and IL-15Rα domains.

FIG. 2 shows an exemplary cassette design for the IL-15/IL-15Rα-Fc fusion protein of the present disclosure compared to a standard reference cassette.

FIG. 3 shows binding of IL-15/IL-15Rα-Fc fusion proteins to the IL-2Rβ (CD122).

FIG. 4 shows the effects of IL-15/IL-15Rα-Fc fusion proteins on NK cell proliferation.

FIG. 5 shows the effects of IL-15/IL-15Rα-Fc fusion proteins on NK cell cytotoxicity.

FIG. 6 shows the effects of IL-15/IL-15Rα-Fc fusion proteins on NK cell degranulation. NK cells were treated with various concentrations of IL-15/IL-15Rα-Fc fusion proteins overnight. NK cell degranulation was analyzed with anti-CD107a antibody and flow cytometry.

FIG. 7A-FIG. 7B show the effects of IL-15/IL-15Rα-Fc fusion proteins on NK and CD8+ T cell signaling. FIG. 7A shows phosphorylation of STAT5 in primary NK cells treated with 0.5 nM of IL-15/IL-15Rα-Fc fusion proteins overnight. FIG. 7B shows phosphorylation of STAT5 in primary CD8+ T cells treated with 0.5 nM of IL-15/IL-15Rα-Fc fusion proteins overnight. STAT5 phosphorylation was analyzed with anti-phospho-STAT5 antibody and flow cytometry.

FIG. 8A shows the effects of treatment with IL-15/IL-15Rα-Fc fusion proteins on tumor growth and metastases in tumor-burdened mice after 15 days. FIG. 8B shows the effects of treatment with IL-15/IL-15Rα-Fc fusion proteins on tumor growth and metastases in tumor-burdened mice after 21 days.

FIG. 9A shows exemplary effects of treatment with IL-15/IL-15Rα-Fc fusion proteins on tumor growth in lungs excised from tumor-burden mice compared to vehicle control. FIG. 9B shows exemplary effects of IL-15/IL-15Rα-Fc fusion proteins on metastases compared to vehicle control. Molecule V1(SEQ ID NOs: 31) is a standard IL-15/IL-15Rα-Fc fusion protein shown for reference.

FIG. 10 shows a Kaplan-Meier survival curve of tumor-burdened mice administered the IL-15/IL-15Rα-Fc fusion protein Molecule Z1 at various concentrations compared to vehicle control. Molecule V1 is a standard IL-15/IL-15Rα-Fc fusion protein shown for reference.

FIG. 11 shows the Tumor Growth Inhibition value (TGI %) on lung metastases of B16F10-Luc mice administered the IL-15/IL-15Rα-Fc fusion protein Molecule Y1 at 0.125, 0.25 and 0.5 mg/kg compared to vehicle control.

### DETAILED DESCRIPTION

### Definitions

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein the term “sequence identity” refers to the percentage of nucleotide or amino acid sequences in a candidate sequence that are identical to the amino acid residues or nucleotides in the specific (parental) sequence, after aligning the sequences and introducing gaps if necessary, to achieve maximum percent identity. Alignment for purposes of determining percent amino acid sequence identity can be achieved in various ways, for instance, using publicly available computer software such as BLAST, BLAST-2, ALIGN, Clustal Omega®, or Megalign (DNASTAR) software. Another approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986).

As used herein, the terms “treat,” “treating,” or “treatment”, and grammatical variants thereof, have the same meaning as commonly understood in the art. In some embodiments, these terms may refer to an approach for obtaining beneficial or desired clinical results. The terms may refer to slowing the onset or rate of development of a condition, disorder or disease, reducing or alleviating symptoms associated with it, generating a complete or partial regression of the condition, or some combination of any of the above. For the purposes of this disclosure, beneficial or desired clinical results include, but are not limited to, reduction or alleviation of symptoms, diminishment of extent of disease, stabilization (e.g., not worsening) of state of disease, delay or slowing of disease progression, amelioration or palliation of the disease state, and remission (whether partial or total), whether detectable or undetectable. “Treat,” “treating,” or “treatment” can also mean prolonging survival relative to expected survival time if not receiving treatment. A subject (e.g., a human) in need of treatment may thus be a subject already afflicted with the disease or disorder in question. The terms “treat,” “treating,” or “treatment” includes inhibition or reduction of an increase in severity of a pathological state or symptoms relative to the absence of treatment and is not necessarily meant to imply complete cessation of the relevant disease or condition.

As used herein, the terms “prevent,” “preventing,” “prevention” and grammatical variants thereof refer to an approach for preventing the development of, or altering the pathology of, a condition or disease. Accordingly, “prevention” may refer to prophylactic or preventive measures. For the purposes of this disclosure, beneficial or desired clinical results include, but are not limited to, prevention or slowing of symptoms, progression or development of a disease, whether detectable or undetectable. A subject (e.g., a human) in need of prevention may thus be a subject not yet afflicted with the disease or disorder in question. The term “prevention” includes slowing the onset of disease relative to the absence of treatment and is not necessarily meant to imply permanent prevention of the relevant disease, disorder or condition. Thus “preventing” or “prevention” of a condition may in certain contexts refer to reducing the risk of developing the condition or preventing or delaying the development of symptoms associated with the condition.

The phrases “pharmaceutically acceptable carrier” or “pharmaceutically acceptable diluent” refers to any material, composition, or vehicle that is physiologically acceptable, i.e., compatible buffer, solvents, dispersion media, coatings, antimicrobial agents, isotonic, absorption delaying agents, solid or liquid filler, excipients, encapsulating material, and the like. The phrase “pharmaceutically acceptable excipient” means an excipient that is useful in preparing a pharmaceutical composition that is generally safe, non-toxic and neither biologically nor otherwise undesirable, and includes an excipient, that is acceptable for human pharmaceutical use as well veterinary use.

The phrase “therapeutically effective amount,” as used herein, refers to an amount of a compound, such as an IL-15/IL-15Rα-Fc fusion protein, that achieves the desired biologic or therapeutic effect, namely an amount that prevents, reduces or ameliorates one or more symptoms of the enumerated diseases being treated or prevented.

As used herein, the term “additional therapeutic” means one or more therapeutics that provide a therapeutic effect or benefit to a subject in need thereof. The additional therapeutic may be a therapeutic agent (e.g., chemotherapy agent) or a medical device.

As used herein, the term “subject” refers to any subject, e.g., a human or a non-human mammal, for whom diagnosis, prognosis, or therapy is desired. The term “subject” may mean a human or non-human mammal affected, likely to be affected, or suspected to be affected with a disease. The terms “subject” and “patient” are used interchangeably herein. In some embodiments, a subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile, fish, or the like.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

The term “antigen” refers to a molecule or a portion of a molecule capable of being bound by an antibody or an antigen-binding fragment thereof and additionally capable of being used in an animal to produce antibodies capable of binding to an epitope of that antigen. An antigen may have one or more epitopes.

The term “activation” when referring to treatment with the IL-15/IL-15Rα-Fc fusion proteins described herein, refers to activation of the immune system, such as activation of NK cells and T cells. Activation of NK cells and T cells by the IL-15/IL-15Rα-Fc fusion proteins described herein results in, for example, increased development, proliferation, survival, cytotoxicity, degranulation, and trafficking. This results in enhanced anti-tumor immunity leading to reduced tumor growth and metastasis.

The term “cytotoxicity” refers to an ability of an immune cell to kill a target cell. In some embodiments, the immune cell is a CD8+ T cell, NK cell, or an NKT cell. In some embodiments, the target cell is a tumor cell or a virally-infected cell.

The term “degranulation” refers to a cellular process that releases cytotoxic effector molecules from secretory vesicles called granules from cells. Degranulation of T cells and NK cells results in release of perforin and granzyme that kill target cells, such as a tumor or virally-infected cells.

### IL-15/IL-15Rα-Fc Fusion Proteins

The present disclosure relates to interleukin-15 (IL-15) agonists and methods of use thereof. One embodiment of the present disclosure provides an IL-15/IL-15 receptor alpha (IL-15Rα)-Fc fusion protein complex including from N-terminus to C-terminus: an IL-15Rα sushi domain, an IL-15 C-terminal domain, an IL-15 N-terminal domain, and an Fc domain. In some embodiments, the IL-15/IL-15 receptor alpha (IL-15Rα)-Fc fusion proteins of the present disclosure are used for the treatment of cancer.

IL-15 is a member of the interleukin 2 (IL-2) superfamily and functions in the development, survival, proliferation, and activation of multiple lymphocyte lineages, including T cells and NK cells. IL-15 binds and signals via a trimeric receptor consisting of a common gamma chain, IL-2R beta (IL-2Rβ) and IL-15 receptor alpha (IL-15Rα). Free IL-15 exists at very low levels in the bloodstream and is often found complexed with IL-15 receptor alpha (IL-15Rα), which improves both the half-life and bioavailability of IL-15. This specific presentation of IL-15 can also improve activation of T, B, and NK cells, which plays a critical role in tumor immunosurveillance and inhibition though early recognition and destruction of malignant cells.

IL-15 may not induce activation-induced cell death (AICD) of T cells or enhance the proliferation, function, or differentiation of immunosuppressive CD4+ T regulatory cells (Tregs). In addition, IL-15 may not induce severe capillary leak syndrome in nonhuman primates (NHP) or humans, a toxicity associated with IL-2 therapy. Thus, IL-15 may activate effector immune cells necessary for tumor control with fewer associated toxicities.

In contrast to recombinant IL-15, IL-15/IL-15 receptor alpha (IL-15Rα)-Fc fusion proteins may not require trans-representation or cell-to-cell contact to induce IL-15-mediated immune responses. They may also have greater potency, bioavailability, and stability than soluble recombinant IL-15 due to their structure.

An illustrative sequence for human IL-15 is provided as SEQ ID NO: 1. See also Uniprot Accession No. P40933.

(SEQ ID NO: 1)
MRISKPHLRS ISIQCYLCLL LNSHFLTEAG IHVFILGCFS
AGLPKTEANW VNVISDLKKI EDLIQSMHID ATLYTESDVH
PSCKVTAMKC FLLELQVISL ESGDASIHDT VENLIILANN
SLSSNGNVTE SGCKECEELE EKNIKEFLQS FVHIVQMFIN
TS.

An illustrative sequence for human IL-15Rα is provided as SEQ ID NO: 2. See also Uniprot Accession No. Q13261.

(SEQ ID NO: 2)
MAPRRARGCRTLGLPALLLLLLLRPPATRGITCPPPMSVEHADIWVKS
YSLYSRERYICNSGFKRKAGTSSLTECVLNKATNVAHWTTPSLKCIRD
PALVHQRPAPPSTVTTAGVTPQPESLSPSGKEPAASSPSSNNTAATTA
AIVPGSQLMPSKSPSTGTTEISSHESSHGTPSQTTAKNWELTASASHQ
PPGVYPQGHSDTTVAISTSTVLLCGLSAVSLLACYLKSRQTPPLASVE
MEAMEALPVTWGTSSRDEDLENCSHHL.

### IL-15 Domains

In some embodiments, the IL-15/IL-15Rα-Fc fusion protein includes one or more IL-15 domains. In some embodiments, the IL-15/IL-15Rα-Fc fusion protein includes an IL-15 C-terminal domain. In some embodiments, the IL-15/IL-15Rα-Fc fusion protein includes an IL-15 N-terminal domain. In some embodiments, the IL-15/IL-15Rα-Fc fusion protein includes an IL-15 C-terminal domain and an IL-15 N-terminal domain. In some embodiments, the IL-15/IL-15Rα-Fc fusion protein includes from N-terminus to C-terminus: an IL-15 C-terminal domain and an IL-15 N-terminal domain.

In some embodiments, the IL-15/IL-15Rα-Fc fusion protein includes an IL-15 C-terminal domain. In some embodiments, the IL-15 C-terminal domain includes an amino acid sequence with at least about 80%, at least about 85%, at least about 90%, at least about 95%, at least about 96%, at least about 97%, or at least about 98% sequence identity to SEQ ID NO: 3 or SEQ ID NO: 4. In some embodiments, the IL-15 C-terminal domain includes an amino acid sequence of SEQ ID NO: 3 or SEQ ID NO: 4 with one or more mutations, such as 1, 2, 3, 4, 5, 6, 7, 8, 9, or 10 mutations. In some embodiments, the IL-15 C-terminal domain includes an amino acid sequence of SEQ ID NO: 3 or SEQ ID NO: 4. In some embodiments, the IL-15 C-terminal domain consists of an amino acid sequence of SEQ ID NO: 3 or SEQ ID NO: 4.

Exemplary IL-15 C-terminal domains are shown in Table 1 below.

TABLE 1

| | | SEQ |
|---|---|---|
| Description | Amino Acid Sequence | ID NO. |
| IL-15 C-term | NVTESGCKECEELEEKNIK | 3 |
| Domain 1 | EFLQSFVHIVQMFINTS | |
| IL-15 C-term | TESGCKECEELEEKNIKEF | 4 |
| Domain 2 | LQSFVHIVQMFINTS | |

In some embodiments, the IL-15/IL-15Rα-Fc fusion protein includes an IL-15 N-terminal domain. In some embodiments, the IL-15 N-terminal domain includes an amino acid sequence with at least about 80%, at least about 85%, at least about 90%, at least about 95%, at least about 96%, at least about 97%, at least about 98%, or at least about 99% sequence identity to any one of SEQ ID NOs: 5-7. In some embodiments, the IL-15 C-terminal domain includes an amino acid sequence of any one of SEQ ID NOs: 5-7 with one or more mutations, such as 1, 2, 3, 4, 5, 6, 7, 8, 9, or 10 mutations. In some embodiments, the IL-15 N-terminal domain includes one or more mutations at residues selected from the group consisting of: V3, N4, S7, D8, K11, D30, D61, E64, N65, 168, L69, N72, and any combination thereof. In some embodiments, the IL-15 N-terminal domain includes one or more mutations selected from the group consisting of: V3A, V3L, N4Q, N4A, N4D, S7D, S7V, D8T, D8A, K11A, D30N, D61A, D61S, D61N, D61E, E64Q, N65D, I68A, I68V, L69A, L69V, N72E, N72A, and any combination thereof. In some embodiments, the IL-15 N-terminal domain includes an N72D mutation. In some embodiments, the IL-15 N-terminal domain includes a D30N mutation, a E64Q mutation, and a N65D mutation. In some embodiments, the IL-15 N-terminal domain includes a N4D mutation and a N65D mutation. In some embodiments, the IL-15 N-terminal domain includes a D30N mutation and a N65D mutation. In some embodiments, the IL-15 N-terminal domain includes an I68A mutation and a L69A mutation. In some embodiments, the IL-15 N-terminal domain includes an I68A mutation and a L69V mutation. In some embodiments, the IL-15 N-terminal domain includes an I68V mutation and a L69A mutation. In some embodiments, the IL-15 N-terminal domain includes an I68V mutation and a L69V mutation. In some embodiments, the IL-15 N-terminal domain includes an I68A mutation and a S7V mutation. In some embodiments, the IL-15 N-terminal domain includes an I68A mutation and a S7D mutation. In some embodiments, the IL-15 N-terminal domain includes a L69A mutation and a S7V mutation. In some embodiments, the IL-15 N-terminal domain includes a L69A mutation and a S7D mutation. In some embodiments, the IL-15 N-terminal domain includes a D61A mutation and a L69A mutation. In some embodiments, the IL-15 N-terminal domain includes a D61A mutation and an I68A mutation. In some embodiments, the IL-15 N-terminal domain includes a D61A mutation and a S7V mutation. In some embodiments, the IL-15 N-terminal domain includes a D61A mutation and a S7D mutation. In some embodiments, the IL-15 N-terminal domain includes a D61N mutation and a L69A mutation. In some embodiments, the IL-15 N-terminal domain includes a D61N mutation and an I68A mutation. In some embodiments, the IL-15 N-terminal domain includes a D61S mutation and a L69A mutation. In some embodiments, the IL-15 N-terminal domain includes a D61S mutation and an I68A mutation.

In some embodiments, the IL-15 N-terminal domain includes an amino acid sequence of SEQ ID NO: 5, SEQ ID NO: 6, or SEQ ID NO: 7. In some embodiments, the IL-15 N-terminal domain consists of an amino acid sequence of SEQ ID NO: 5, SEQ ID NO: 6, or SEQ ID NO: 7.

Exemplary IL-15 N-terminal domains are shown in Table 2 below.

TABLE 2

| | | SEQ |
|---|---|---|
| Description | Amino Acid Sequence | ID NO. |
| Design 1: | NWVNVISDLKKIEDLIQSMHIDATLYT | 5 |
| Nterm1 | ESDVHPSCKVTAMKCFLLELQVISLES | |
| | GDASIHDTVENLIILANNSLSSNG | |
| Design 2: | NWVNVISDLKKIEDLIQSMHIDATLYT | 6 |
| Nterm2 | ESDVHPSCKVTAMKCFLLELQVISLES | |
| | GDASIHDTVENLIILANNSLSSNGNV | |
| Design 3: | NWVNVISDLKKIEDLIQSMHIDATLYT | 7 |
| N72D | ESDVHPSCKVTAMKCFLLELQVISLES | |
| | GDASIHDTVENLIILANDSLSSNGNV | |

### IL-15Rα Domains

In some embodiments, the IL-15/IL-15Rα-Fc fusion protein includes an IL-15Rα domain. In some embodiments, the IL-15/IL-15Rα-Fc fusion protein includes an IL-15Rα sushi domain. In some embodiments, the IL-15Rα sushi domain includes an amino acid sequence with at least about 80%, at least about 85%, at least about 90%, at least about 95%, at least about 96%, at least about 97%, at least about 98%, or at least about 99% sequence identity to any one of SEQ ID NOs: 8-11. In some embodiments, the IL-15Rα sushi domain includes an amino acid sequence of any one of SEQ ID NOs: 8-11 with one or more mutations, such as 1, 2, 3, 4, 5, 6, 7, 8, 9, or 10 mutations. In some embodiments, the IL-15Rα sushi domain includes an amino acid sequence of any one of SEQ ID NOs: 8-11. In some embodiments, the IL-15Rα sushi domain consists of an amino acid sequence of any one of SEQ ID NOs: 8-11.

Exemplary IL-15Rα sushi domains are shown in Table 3 below.

TABLE 3

| | | SEQ |
|---|---|---|
| Description | Amino Acid Sequence | ID NO. |
| Sushi domain L | ITCPPPMSVEHADIWVKSYSLYSRE | 8 |
| | RYICNSGFKRKAGTSSLTECVLNKA | |
| | TNVAHWTTPSLKCIRDPALVHQRPA | |
| | PPS | |
| Sushi domain M | ITCPPPMSVEHADIWVKSYSLYSRE | 9 |
| | RYICNSGFKRKAGTSSLTECVLNKA | |
| | TNVAHWTTPSLKCIRDPALVHQR | |
| Sushi domain S | ITCPPPMSVEHADIWVKSYSLYSRE | 10 |
| | RYICNSGFKRKAGTSSLTECVLNKA | |
| | TNVAHWTTPSLKCIRDPA | |
| Sushi domain VS | ITCPPPMSVEHADIWVKSYSLYSRE | 11 |
| | RYICNSGFKRKAGTSSLTECVLNKA | |
| | TNVAHWTTPSLKCIR | |

### Fc Domains

In some embodiments, the IL-15/IL-15Rα-Fc fusion protein includes an Fc domain. In some embodiments, the IL-15/IL-15Rα-Fc fusion protein includes an IgG1 Fc domain. In some embodiments, the IL-15/IL-15Rα-Fc fusion protein includes an IgG2 Fc domain. In some embodiments, the IL-15/IL-15Rα-Fc fusion protein includes an IgG3 Fc domain. In some embodiments, the IL-15/IL-15Rα-Fc fusion protein includes an IgG4 Fc domain.

In some embodiments, the IL-15/IL-15Rα-Fc fusion protein includes an IgG1 Fc domain. In some embodiments, the IgG1 Fc domain includes an amino acid sequence with at least about 80%, at least about 85%, at least about 90%, at least about 95%, at least about 96%, at least about 97%, at least about 98%, or at least about 99% sequence identity to any one of SEQ ID NOs: 12-16. In some embodiments, the IgG1 Fc domain includes an amino acid sequence of any one of SEQ ID NOs: 12-16 with one or more mutations, such as 1, 2, 3, 4, 5, 6, 7, 8, 9, or 10 mutations. In some embodiments, the IgG1 Fc domain includes an amino acid sequence of any one of SEQ ID NOs: 12-16. In some embodiments, the IgG1 Fc domain consists of an amino acid sequence of any one of SEQ ID NOs: 12-16.

In some embodiments, the IL-15/IL-15Rα-Fc fusion protein includes an IgG1 Fc domain. In some embodiments, the IgG1 Fc domain includes an amino acid sequence with at least about 80%, at least about 85%, at least about 90%, at least about 95%, at least about 96%, at least about 97%, at least about 98%, or at least about 99% sequence identity to SEQ ID NO: 12. In some embodiments, the IgG1 Fc domain includes an amino acid sequence of SEQ ID NO: 12 with one or more mutations, such as 1, 2, 3, 4, 5, 6, 7, 8, 9, or 10 mutations. In some embodiments, the IgG1 Fc domain includes an amino acid sequence of SEQ ID NO: 12. In some embodiments, the IgG1 Fc domain consists of an amino acid sequence of SEQ ID NO: 12.

In some embodiments, the IL-15/IL-15Rα-Fc fusion protein includes an IgG4 Fc domain. In some embodiments, the IgG4 Fc domain includes an amino acid sequence with at least about 80%, at least about 85%, at least about 90%, at least about 95%, at least about 96%, at least about 97%, at least about 98%, or at least about 99% sequence identity to SEQ ID NO: 17. In some embodiments, the IgG4 Fc domain includes an amino acid sequence of SEQ ID NO: 17 with one or more mutations, such as 1, 2, 3, 4, 5, 6, 7, 8, 9, or 10 mutations. In some embodiments, the IgG4 Fc domain includes an amino acid sequence of SEQ ID NO: 17. In some embodiments, the IgG4 Fc domain consists of an amino acid sequence of SEQ ID NO: 17.

Exemplary Fc domains are shown in Table 4 below. Mutations in the IgG1 Fc domain are represented using bolded and underlined text.

TABLE 4

| | | SEQ |
|---|---|---|
| Description | Amino Acid Sequence | ID NO. |
| Human IgG1 | EPKSCDKTHTCPPCPAPELLGGPSVFLFPPKPKDTLMISR | 12 |
| | TPEVTCVVVDVSHEDPEVKFNWYVDGVEVHNAKTKP | |
| | REEQYNSTYRVVSVLTVLHQDWLNGKEYKCKVSNKA | |
| | LPAPIEKTISKAKGQPREPQVYTLPPSRDELTKNQVSLT | |
| | CLVKGFYPSDIAVEWESNGQPENNYKTTPPVLDSDGSF | |
| | FLYSKLTVDKSRWQQGNVFSCSVMHEALHNHYTQKSL | |
| | SLSPGK | |
| Human IgG1 | EPKSCDKTHTCPPCPAPELLGGPSVFLFPPKPKDTLMISR | 13 |
| (EFL1, N297A) | TPEVTCVVVDVSHEDPEVKFNWYVDGVEVHNAKTKP | |
| | REEQYASTYRVVSVLTVLHQDWLNGKEYKCKVSNKA | |
| | LPAPIEKTISKAKGQPREPQVYTLPPSRDELTKNQVSLT | |
| | CLVKGFYPSDIAVEWESNGQPENNYKTTPPVLDSDGSF | |
| | FLYSKLTVDKSRWQQGNVFSCSVMHEALHNHYTQKSL | |
| | SLSPGK | |
| Human IgG1 | EPKSCDKTHTCPPCPAPEAAGGPSVFLFPPKPKDTLMIS | 14 |
| (EFL2, LALA) | RTPEVTCVVVDVSHEDPEVKFNWYVDGVEVHNAKTK | |
| | PREEQYNSTYRVVSVLTVLHQDWLNGKEYKCKVSNK | |
| | ALPAPIEKTISKAKGQPREPQVYTLPPSRDELTKNQVSL | |
| | TCLVKGFYPSDIAVEWESNGQPENNYKTTPPVLDSDGS | |
| | FFLYSKLTVDKSRWQQGNVFSCSVMHEALHNHYTQKS | |
| | LSLSPGK | |
| Human IgG1 | EPKSCDKTHTCPPCPAPEAAGGPSVFLFPPKPKDTLMIS | 15 |
| (EFL3, LALAPG) | RTPEVTCVVVDVSHEDPEVKFNWYVDGVEVHNAKTK | |
| | PREEQYNSTYRVVSVLTVLHQDWLNGKEYKCKVSNK | |
| | ALGAPIEKTISKAKGQPREPQVYTLPPSRDELTKNQVSL | |
| | TCLVKGFYPSDIAVEWESNGQPENNYKTTPPVLDSDGS | |
| | FFLYSKLTVDKSRWQQGNVFSCSVMHEALHNHYTQKS | |
| | LSLSPGK | |
| Human IgG1 | EPKSCDKTHTCPPCPAPELLGGPSVFLFPPKPKDTLYITR | 16 |
| (HLE)-YTE | EPEVTCVVVDVSHEDPEVKFNWYVDGVEVHNAKTKP | |
| | REEQYNSTYRVVSVLTVLHQDWLNGKEYKCKVSNKA | |
| | LPAPIEKTISKAKGQPREPQVYTLPPSRDELTKNQVSLT | |
| | CLVKGFYPSDIAVEWESNGQPENNYKTTPPVLDSDGSF | |
| | FLYSKLTVDKSRWQQGNVFSCSVMHEALHNHYTQKSL | |
| | SLSPGK | |
| Human IgG4 | ESKYGPPCPSCPAPEFLGGPSVFLFPPKPKDTLMISRTPE | 17 |
| | VTCVVVDVSQEDPEVQFNWYVDGVEVHNAKTKPREE | |
| | QFNSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |

### Linkers

In some embodiments, the IL-15/IL-15Rα-Fc fusion protein includes one or more linkers. As used herein, the term “linker” refers to a polypeptide sequence that joins two protein domains together. In some embodiments, the IL-15/IL-15Rα-Fc fusion protein includes one linker. In some embodiments, the IL-15/IL-15Rα-Fc fusion protein includes two linkers. In some embodiments, the IL-15/IL-15Rα-Fc fusion protein includes three linkers. In some embodiments, the IL-15/IL-15Rα-Fc fusion protein does not include a linker.

In some embodiments, the IL-15/IL-15Rα-Fc fusion protein includes one or more linkers including at least one amino acid. In some embodiments, the IL-15/IL-15Rα-Fc fusion protein includes one or more linkers including 1 amino acid, 2 amino acids, 3 amino acids, 4 amino acids, 5 amino acids, 6 amino acids, 7 amino acids, 8 amino acids, 9 amino acids, 10 amino acids, 11 amino acids, 12 amino acids, 13 amino acids, 14 amino acids, or more.

In some embodiments, the IL-15/IL-15Rα-Fc fusion protein includes a linker between the IL-15Rα sushi domain and the IL-15 C-terminal domain. In some embodiments, the IL-15/IL-15Rα-Fc fusion protein includes a linker between the IL-15 C-terminal domain and the IL-15 N-terminal domain. In some embodiments, the IL-15/IL-15Rα-Fc fusion protein includes a linker between the IL-15 N-terminal domain and the Fc domain. In some embodiments, the IL-15/IL-15Rα-Fc fusion protein includes a linker between the IL-15 C-terminal domain and the IL-15 N-terminal domain; and a linker between the IL-15 N-terminal domain and the Fc domain.

In some embodiments, the IL-15/IL-15Rα-Fc fusion protein includes one or more linkers, and the one or more linkers includes an amino acid sequence of any one of the linkers listed in Table 5. In some embodiments, the IL-15/IL-15Rα-Fc fusion protein includes one or more linkers, and the one or more linkers includes an amino acid sequence of GS, GG, GGS, GGGS (SEQ ID NO: 18), GGGGS (SEQ ID NO: 19), or any combination thereof. In some embodiments, the IL-15/IL-15Rα-Fc fusion protein includes one or more linkers, and the one or more linkers includes an amino acid sequence of GGGGS (SEQ ID NO: 19). In some embodiments, the IL-15/IL-15Rα-Fc fusion protein includes one or more linkers, and the one or more linkers consists of an amino acid sequence of GGGGS (SEQ ID NO: 19).

Exemplary linkers are shown in Table 5 below.

TABLE 5

| Description | Amino Acid Sequence |
|---|---|
| Linker 1 | GS |
| Linker 2 | GG |
| Linker 3 | GGS |
| Linker 4 | GGGS |
| Linker 5 | GGGGS |

### Exemplary IL-15/IL-15Rα-Fc Fusion Proteins

In some embodiments, the IL-15/IL-15Rα-Fc fusion protein includes from N-terminus to C-terminus, an IL-15Rα sushi domain, an IL-15 C-terminal domain, an IL-15 N-terminal domain, and an Fc domain. In some embodiments, the IL-15/IL-15Rα-Fc fusion protein includes from N-terminus to C-terminus, an IL-15Rα sushi domain, an IL-15 C-terminal domain, a linker, an IL-15 N-terminal domain, and an Fc domain. In some embodiments, the IL-15/IL-15Rα-Fc fusion protein includes from N-terminus to C-terminus, an IL-15Rα sushi domain, an IL-15 C-terminal domain, an IL-15 N-terminal domain, a linker, and an Fc domain. In some embodiments, the IL-15/IL-15Rα-Fc fusion protein includes from N-terminus to C-terminus, an IL-15Rα sushi domain, an IL-15 C-terminal domain, a linker, an IL-15 N-terminal domain, a linker, and an Fc domain.

In some embodiments, the IL-15/IL-15Rα-Fc fusion protein includes an amino acid sequence with at least about 80%, at least about 85%, at least about 90%, at least about 95%, at least about 96%, at least about 97%, at least about 98%, or at least about 99% sequence identity to any one of SEQ ID NOs: 20-30. In some embodiments, the IL-15/IL-15Rα-Fc fusion protein includes an amino acid sequence of any one of SEQ ID NOs: 20-30 with one or more mutations, such as 1, 2, 3, 4, 5, 6, 7, 8, 9, or 10 mutations. In some embodiments, the IL-15/IL-15Rα-Fc fusion protein includes an amino acid sequence of any one of SEQ ID NOs: 20-30. In some embodiments, the IL-15/IL-15Rα-Fc fusion protein consists of an amino acid sequence of any one of SEQ ID NOs: 20-30.

In some embodiments, the IL-15/IL-15Rα-Fc fusion protein includes an amino acid sequence of SEQ ID NO: 24 with one or more mutations selected from the group consisting of: V3A, V3L, N4A, N4D, N4Q, S7D, S7V, D8T, D8A, K11A, D30N, D61A, D61S, D61N, D61E, E64Q, N65D, I68A, I68V, L69A, L69V, N72E, and N72A.

In some embodiments, the IL-15/IL-15Rα-Fc fusion protein includes an amino acid sequence with at least about 80%, at least about 85%, at least about 90%, at least about 95%, at least about 96%, at least about 97%, at least about 98%, or at least about 99% sequence identity to SEQ ID NO: 28. In some embodiments, the IL-15/IL-15Rα-Fc fusion protein includes an amino acid sequence of SEQ ID NO: 28 with one or more mutations, such as 1, 2, 3, 4, 5, 6, 7, 8, 9, or 10 mutations. In some embodiments, the IL-15/IL-15Rα-Fc fusion protein includes an amino acid sequence of SEQ ID NO: 28. In some embodiments, the IL-15/IL-15Rα-Fc fusion protein consists of an amino acid sequence of SEQ ID NO: 28.

In some embodiments, the IL-15/IL-15Rα-Fc fusion protein includes an amino acid sequence of SEQ ID NO: 28 with one or more mutations selected from the group consisting of: N189Q and G190A.

In some embodiments, the IL-15/IL-15Rα-Fc fusion protein includes an amino acid sequence of SEQ ID NO: 28 with one or more linkers, and the one or more linkers include an amino acid sequence of GS, GG, GGS, GGGS, or GGGGS.

In some embodiments, the IL-15/IL-15Rα-Fc fusion protein includes an amino acid sequence of SEQ ID NO: 30 with one or more mutations selected from the group consisting of: N189Q and G190A.

Exemplary IL-15/IL-15Rα-Fc fusion proteins are illustrated in Table 6 below.

TABLE 6

| | | | SEQ |
|---|---|---|---|
| ID | Description | Amino Acid Sequence | ID NO. |
| Molecule N1 | IL15Rα- | ITCPPPMSVEHADIWVKSYSLYSRERYICNSGFKRKA | 20 |
| | L/IL15 DS1 | GTSSLTECVLNKATNVAHWTTPSLKCIRDPALVHQR | |
| | | PAPPSNVTESGCKECEELEEKNIKEFLQSFVHIVQMFI | |
| | | NTSGGGGSNWVNVISDLKKIEDLIQSMHIDATLYTES | |
| | | DVHPSCKVTAMKCFLLELQVISLESGDASIHDTVENL | |
| | | IILANNSLSSNGGGGGSEPKSCDKTHTCPPCPAPELLG | |
| | | GPSVFLFPPKPKDTLMISRTPEVTCVVVDVSHEDPEV | |
| | | KFNWYVDGVEVHNAKTKPREEQYNSTYRVVSVLTV | |
| | | LHQDWLNGKEYKCKVSNKALPAPIEKTISKAKGQPR | |
| | | EPQVYTLPPSRDELTKNQVSLTCLVKGFYPSDIAVE | |
| | | WESNGQPENNYKTTPPVLDSDGSFFLYSKLTVDKSR | |
| | | WQQGNVFSCSVMHEALHNHYTQKSLSLSPGK | |
| Molecule O1 | Molecule N1 | ITCPPPMSVEHADIWVKSYSLYSRERYICNSGFKRKA | 21 |
| | w/ EFL1 | GTSSLTECVLNKATNVAHWTTPSLKCIRDPALVHQR | |
| | | PAPPSNVTESGCKECEELEEKNIKEFLQSFVHIVQMFI | |
| | | NTSGGGGSNWVNVISDLKKIEDLIQSMHIDATLYTES | |
| | | DVHPSCKVTAMKCFLLELQVISLESGDASIHDTVENL | |
| | | IILANNSLSSNGGGGGSEPKSCDKTHTCPPCPAPELLG | |
| | | GPSVFLFPPKPKDTLMISRTPEVTCVVVDVSHEDPEV | |
| | | KFNWYVDGVEVHNAKTKPREEQYASTYRVVSVLTV | |
| | | LHQDWLNGKEYKCKVSNKALPAPIEKTISKAKGQPR | |
| | | EPQVYTLPPSRDELTKNQVSLTCLVKGFYPSDIAVE | |
| | | WESNGQPENNYKTTPPVLDSDGSFFLYSKLTVDKSR | |
| | | WQQGNVFSCSVMHEALHNHYTQKSLSLSPGK | |
| Molecule P1 | IL15Rα- | ITCPPPMSVEHADIWVKSYSLYSRERYICNSGFKRKA | 22 |
| | S/IL15 DS1 | GTSSLTECVLNKATNVAHWTTPSLKCIRDPANVTES | |
| | | GCKECEELEEKNIKEFLQSFVHIVQMFINTSGGGGSN | |
| | | WVNVISDLKKIEDLIQSMHIDATLYTESDVHPSCKVT | |
| | | AMK | |
| | | CFLLELQVISLESGDASIHDTVENLIILANNSLSSNGG | |
| | | GGGSEPKSCDKTHTCPPCPAPELLGGPSVFLFPPKPK | |
| | | DTLMISRTPEVTCVVVDVSHEDPEVKFNWYVDGVE | |
| | | VHNAKTKPREEQYNSTYRVVSVLTVLHQDWLNGKE | |
| | | YKCKVSNKALPAPIEKTISKAKGQPREPQVYTLPPSR | |
| | | DELTKNQVSLTCLVKGFYPSDIAVEWESNGQPENNY | |
| | | KTTPPVLDSDGSFFLYSKLTVDKSRWQQGNVFSCSV | |
| | | MHEALHNHYTQKSLSLSPGK | |
| Molecule Q1 | IL15Rα- | ITCPPPMSVEHADIWVKSYSLYSRERYICNSGFKRKA | 23 |
| | L/IL15 DS2 | GTSSLTECVLNKATNVAHWTTPSLKCIRDPALVHQR | |
| | | PAPPSTESGCKECEELEEKNIKEFLQSFVHIVQMFINT | |
| | | SGGGGSNWVNVISDLKKIEDLIQSMHIDATLYTESDV | |
| | | HPSCKVTAMKCFLLELQVISLESGDASIHDTVENLIIL | |
| | | ANNSLSSNGNVGGGGSEPKSCDKTHTCPPCPAPELL | |
| | | GGPSVFLFPPKPKDTLMISRTPEVTCVVVDVSHEDPE | |
| | | VKFNWYVDGVEVHNAKTKPREEQYNSTYRVVSVLT | |
| | | VLHQDWLN | |
| | | GKEYKCKVSNKALPAPIEKTISKAKGQPREPQVYTLP | |
| | | PSRDELTKNQVSLTCLVKGFYPSDIAVEWESNGQPE | |
| | | NNYKTTPPVLDSDGSFFLYSKLTVDKSRWQQGNVFS | |
| | | CSVMHEALHNHYTQKSLSLSPGK | |
| Molecule R1 | IL15Rα- | ITCPPPMSVEHADIWVKSYSLYSRERYICNSGFKRKA | 24 |
| | M/IL15 DS2 | GTSSLTECVLNKATNVAHWTTPSLKCIRDPALVHQR | |
| | | TESGCKECEELEEKNIKEFLQSFVHIVQMFINTSGGG | |
| | | GSNWVNVISDLKKIEDLIQSMHIDATLYTESDVHPSC | |
| | | KVTAMKCFLLELQVISLESGDASIHDTVENLIILANNS | |
| | | LSSNGNVGGGGSEPKSCDKTHTCPPCPAPELLGGPSV | |
| | | FLFPPKPKDTLMISRTPEVTCVVVDVSHEDPEVKFN | |
| | | WYVDGVEVHNAKTKPREEQYNSTYRVVSVLTVLH | |
| | | QDWLNGKEYKCKVSNKALPAPIEKTISKAKGQPREP | |
| | | QVYTLPPSRDELTKNQVSLTCLVKGFYPSDIAVEWE | |
| | | SNGQPENNYKTTPPVLDSDGSFFLYSKLTVDKSRWQ | |
| | | QGNVFSCSVMHEALHNHYTQKSLSLSPGK | |
| Molecule S1 | Molecule R1 | ITCPPPMSVEHADIWVKSYSLYSRERYICNSGFKRKA | 25 |
| | w/ N72D | GTSSLTECVLNKATNVAHWTTPSLKCIRDPALVHQR | |
| | | TESGCKECEELEEKNIKEFLQSFVHIVQMFINTSGGG | |
| | | GSNWVNVISDLKKIEDLIQSMHIDATLYTESDVHPSC | |
| | | KVTAMKCFLLELQVISLESGDASIHDTVENLIILANDS | |
| | | LSSNGNVGGGGSEPKSCDKTHTCPPCPAPELLGGPSV | |
| | | FLFPPKPKDTLMISRTPEVTCVVVDVSHEDPEVKFN | |
| | | WYVDGVEVHNAKTKPREEQYNSTYRVVSVLTVLH | |
| | | QDWLNGKEYKCKVSNKALPAPIEKTISKAKGQPREP | |
| | | QVYTLPPSRDELTKNQVSLTCLVKGFYPSDIAVEWE | |
| | | SNGQPENNYKTTPPVLDSDGSFFLYSKLTVDKSRWQ | |
| | | QGNVFSCSVMHEALHNHYTQKSLSLSPGK | |
| Molecule T1 | Molecule R1 | ITCPPPMSVEHADIWVKSYSLYSRERYICNSGFKRKA | 26 |
| | w/ EFL1 | GTSSLTECVLNKATNVAHWTTPSLKCIRDPALVHQR | |
| | | TESGCKECEELEEKNIKEFLQSFVHIVQMFINTSGGG | |
| | | GSNWVNVISDLKKIEDLIQSMHIDATLYTESDVHPSC | |
| | | KVTAMKCFLLELQVISLESGDASIHDTVENLIILANNS | |
| | | LSSNGNVGGGGSEPKSCDKTHTCPPCPAPELLGGPSV | |
| | | FLFPPKPKDTLMISRTPEVTCVVVDVSHEDPEVKFN | |
| | | WYVDGVEVHNAKTKPREEQYASTYRVVSVLTVLH | |
| | | QDWLNGKEYKCKVSNKALPAPIEKTISKAKGQPREP | |
| | | QVYTLPPSRDELTKNQVSLTCLVKGFYPSDIAVEWE | |
| | | SNGQPENNYKTTPPVLDSDGSFFLYSKLTVDKSRWQ | |
| | | QGNVFSCSVMHEALHNHYTQKSLSLSPGK | |
| Molecule Y1 | Molecule R1 | ITCPPPMSVEHADIWVKSYSLYSRERYICNSGFKRKA | 27 |
| | w/ EFL2 | GTSSLTECVLNKATNVAHWTTPSLKCIRDPALVHQR | |
| | | TESGCKECEELEEKNIKEFLQSFVHIVQMFINTSGGG | |
| | | GSNWVNVISDLKKIEDLIQSMHIDATLYTESDVHPSC | |
| | | KVTAMKCFLLELQVISLESGDASIHDTVENLIILANNS | |
| | | LSSNGNVGGGGSEPKSCDKTHTCPPCPAPEAAGGPS | |
| | | VFLFPPKPKDTLMISRTPEVTCVVVDVSHEDPEVKFN | |
| | | WYVDGVEVHNAKTKPREEQYNSTYRVVSVLTVLH | |
| | | QDWLNGKEYKCKVSNKALPAPIEKTISKAKGQPREP | |
| | | QVYTLPPSRDELTKNQVSLTCLVKGFYPSDIAVEWE | |
| | | SNGQPENNYKTTPPVLDSDGSFFLYSKLTVDKSRWQ | |
| | | QGNVFSCSVMHEALHNHYTQKSLSLSPGK | |
| Molecule Z1 | Molecule R1 | ITCPPPMSVEHADIWVKSYSLYSRERYICNSGFKRKA | 28 |
| | w/ EFL3 | GTSSLTECVLNKATNVAHWTTPSLKCIRDPALVHQR | |
| | | TESGCKECEELEEKNIKEFLQSFVHIVQMFINTSGGG | |
| | | GSNWVNVISDLKKIEDLIQSMHIDATLYTESDVHPSC | |
| | | KVTAMKCFLLELQVISLESGDASIHDTVENLIILANNS | |
| | | LSSNGNVGGGGSEPKSCDKTHTCPPCPAPEAAGGPS | |
| | | VFLFPPKPKDTLMISRTPEVTCVVVDVSHEDPEVKFN | |
| | | WYVDGVEVHNAKTKPREEQYNSTYRVVSVLTVLH | |
| | | QDWLNGKEYKCKVSNKALGAPIEKTISKAKGQPREP | |
| | | QVYTLPPSRDELTKNQVSLTCLVKGFYPSDIAVEWE | |
| | | SNGQPENNYKTTPPVLDSDGSFFLYSKLTVDKSRWQ | |
| | | QGNVFSCSVMHEALHNHYTQKSLSLSPGK | |
| Molecule W1 | Molecule R1 | ITCPPPMSVEHADIWVKSYSLYSRERYICNSGFKRKA | 29 |
| | w/o 2nd G4S | GTSSLTECVLNKATNVAHWTTPSLKCIRDPALVHQR | |
| | | TESGCKECEELEEKNIKEFLQSFVHIVQMFINTSGGG | |
| | | GSNWVNVISDLKKIEDLIQSMHIDATLYTESDVHPSC | |
| | | KVTAMKCFLLELQVISLESGDASIHDTVENLIILANNS | |
| | | LSSNGNVEPKSCDKTHTCPPCPAPELLGGPSVFLFPP | |
| | | KPKDTLMISRTPEVTCVVVDVSHEDPEVKFNWYVD | |
| | | GVEVHNAKTKPREEQYNSTYRVVSVLTVLHQDWLN | |
| | | GKEYKCKVSNKALPAPIEKTISKAKGQPREPQVYTLP | |
| | | PSRDELTKNQVSLTCLVKGFYPSDIAVEWESNGQPE | |
| | | NNYKTTPPVLDSDGSFFLYSKLTVDKSRWQQGNVFS | |
| | | CSVMHEALHNHYTQKSLSLSPGK | |
| Molecule X1 | Molecule W1 | ITCPPPMSVEHADIWVKSYSLYSRERYICNSGFKRKA | 30 |
| | w/ EFL3 | GTSSLTECVLNKATNVAHWTTPSLKCIRDPALVHQR | |
| | | TESGCKECEELEEKNIKEFLQSFVHIVQMFINTSGGG | |
| | | GSNWVNVISDLKKIEDLIQSMHIDATLYTESDVHPSC | |
| | | KVTAMKCFLLELQVISLESGDASIHDTVENLIILANNS | |
| | | LSSNGNVEPKSCDKTHTCPPCPAPEAAGGPSVFLFPP | |
| | | KPKDTLMISRTPEVTCVVVDVSHEDPEVKFNWYVD | |
| | | GVEVHNAKTKPREEQYNSTYRVVSVLTVLHQDWLN | |
| | | GKEYKCKVSNKALGAPIEKTISKAKGQPREPQVYTL | |
| | | PPSRDELTKNQVSLTCLVKGFYPSDIAVEWESNGQPE | |
| | | NNYKTTPPVLDSDGSFFLYSKLTVDKSRWQQGNVFS | |
| | | CSVMHEALHNHYTQKSLSLSPGK | |
| Molecule V1 | / | ITCPPPMSVEHADIWVKSYSLYSRERYICNSGFKRKA | 31 |
| | | GTSSLTECVLNKATNVAHWTTPSLKCIRDPALVHQR | |
| | | PAPPSGGSGGGGSGGGSGGGGSGGNWVNVISDLKKI | |
| | | EDLIQSMHIDATLYTESDVHPSCKVTAMKCFLLELQ | |
| | | VISLESGDASIHDTVENLIILANDSLSSNGNVTESGCK | |
| | | ECEELEEKNIKEFLQSFVHIVQMFINTSGGGGSEPKSC | |
| | | DKTHTCPPCPAPELLGGPSVFLFPPKPKDTLMISRTPE | |
| | | VTCVVVDVSHEDPEVKFNWYVDGVEVHNAKTKPR | |
| | | EEQYNSTYRVVSVLTVLHQDWLNGKEYKCKVSNK | |
| | | ALPAPIEKTISKAKGQPREPQVYTLPPSRDELTKNQV | |
| | | SLTCLVKGFYPSDIAVEWESNGQPENNYKTTPPVLD | |
| | | SDGSFFLYSKLTVDKSRWQQGNVFSCSVMHEALHN | |
| | | HYTQKSLSLSPGK | |

### Pharmaceutical Compositions

In some embodiments, the disclosure provides a pharmaceutical composition including an IL-15/IL-15Rα-Fc fusion protein for treating or preventing disease in a subject. In some embodiments, the pharmaceutical composition including the IL-15/IL-15Rα-Fc fusion protein is used for the treatment of cancer.

In some embodiments, the IL-15/IL-15Rα-Fc fusion proteins described herein are formulated as one or more pharmaceutical compositions. In some embodiments, the pharmaceutical composition includes an IL-15/IL-15Rα-Fc fusion protein and a pharmaceutically acceptable carrier, diluent, or excipient.

The pharmaceutical compositions described herein are formulated according to known methods to prepare pharmaceutically useful compositions, and the IL-15/IL-15Rα-Fc fusion protein is combined in a mixture with a pharmaceutically acceptable carrier, diluent or excipient. Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994. The precise nature of the carrier, diluent, or excipient will depend on the route of administration, which may be oral, or by injection, e.g., cutaneous, subcutaneous, or intravenous.

In some embodiments, the pharmaceutical compositions described herein are formulated in one of the following dosage forms: an intravenous dosage form, an intramuscular dosage form, an intraperitoneal dosage form, a subcutaneous dosage form, an oral dosage form, an intranasal dosage form, a suppository dosage form, an intradermal dosage form, or a topical dosage form. In some embodiments, the pharmaceutical compositions described herein are formulated in an intravenous dosage form. In some embodiments, the pharmaceutical compositions described herein are formulated in a subcutaneous dosage form. In some embodiments, the pharmaceutical compositions described herein are formulated in an oral dosage form.

### Therapeutic Methods and Uses

In some embodiments, the IL-15/IL-15Rα-Fc fusion protein or pharmaceutical composition thereof is used in the prevention and/or treatment of diseases, disorders, and conditions in a subject. In some embodiments, the IL-15/IL-15Rα-Fc fusion protein or pharmaceutical composition thereof is used to prevent or treat cancer in a subject.

### Methods of Making and Purifying

Also provided herein are methods for making and/or purifying the IL-15/IL-15Rα-Fc fusion proteins described herein. In some embodiments, the IL-15/IL-15Rα-Fc fusion proteins are produced by recombinant expression in a host cell. The term “host cell” refers to a cell which can support the replication or expression of a nucleic acid (such as an expression vector) encoding an IL-15/IL-15Rα-Fc fusion protein. Host cells can be prokaryotic cells, such as E. coli or eukaryotic cells (e.g., yeast, insect, amphibian, bird, or mammalian cells). For example, immortalized cell lines such as Sf9, HEK293, CHO-K1, HeLa are often used as host cells. Creation and isolation of host cell lines including a nucleic acid or capable of producing an IL-15/IL-15Rα-Fc fusion protein can be accomplished using standard techniques known in the art.

In some embodiments, the methods include preparing a nucleic acid (e.g., an expression vector) encoding an IL-15/IL-15Rα-Fc fusion protein. In some embodiments, the methods include contacting a host cell with the nucleic acid (e.g., an expression vector) encoding the IL-15/IL-15Rα-Fc fusion protein. In some embodiments, the methods include introducing the nucleic acid into the host cell, for example by transfection, viral transduction (e.g., using a lentiviral or AAV vector), direct microinjection, particle bombardment, etc.

The IL-15/IL-15Rα-Fc fusion protein can be produced by culturing a host cell under conditions under which the fusion protein is expressed, and recovering the fusion protein. Culture conditions for producing recombinant proteins using various host cells. In some embodiments, the host cell can be maintained in culture medium at 95° C. with 5% CO₂ atmosphere for a period of time sufficient to express the fusion protein.

When an IL-15/IL-15Rα-Fc fusion protein is expressed using recombinant techniques in a host cell, it is advantageous to separate (or purify) the desired protein away from other components, such as host cell factors, in order to obtain preparations that are of high purity or are substantially homogeneous. Purification can be accomplished by methods well known in the art, such as centrifugation techniques, extraction techniques, chromatographic and fractionation techniques (e.g. size separation by gel filtration, charge separation by ion-exchange column, hydrophobic interaction chromatography, reverse phase chromatography, chromatography on silica or cation-exchange resins such as DEAE and the like, chromatofocusing, and Protein A Sepharose chromatography to remove contaminants), and precipitation techniques (e.g., ethanol precipitation or ammonium sulfate precipitation). Any number of biochemical purification techniques can be used to increase the purity of an IL-15/IL-15Rα-Fc fusion protein.

### EXAMPLES

### Example 1: Design and Generation of IL-15/IL-15Rα-Fc Fusion Proteins

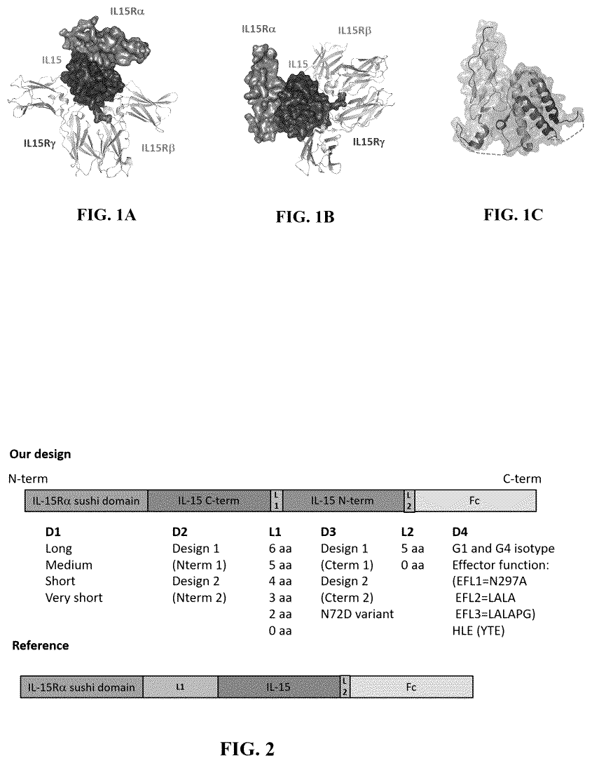

A structure-based design strategy was used to generate a stable complex of Interleukin-15 (IL-15) and IL-15 Receptor alpha (IL-15/IL-15Rα) with a unique sequence series to improve therapeutic potential. Structural analysis using a 3D x-ray diffraction (XRD) structure of the IL-15/IL-15Rα ligand-receptor complex (FIG. 1) was used for rational design of a stable complex of IL-15/IL-15Rα.

FIG. 1A and FIG. 1B show combined ribbon structure and surface view of an IL-15 quaternary complex from different perspectives. FIG. 1C shows the interface between IL-15 and IL-15Rα within the quaternary complex. The dashed lines indicate where the linkers were placed to join the IL-15 and IL-15Rα domains.

A minimal stable complex of IL-15/IL-15Rα sushi domain was identified. This single polypeptide chain chimeric sequence includes from N-terminus to C-terminus: an IL-15Rα sushi domain, a C-terminal domain of IL-15, an N-terminal domain of IL-15, and an Fc domain. IL-15/IL-15Rα-Fc variants were then generated and tested for functionality and efficacy (FIG. 2), which included four different lengths of IL-15Rα sushi domain (D1 of FIG. 2); two different dividing points to separate the IL-15 N-terminal domain from the IL-15 C-terminal domain (D2 and D3 of FIG. 2); and various Fc domains (D4 of FIG. 2).

Linker lengths were also varied between the IL-15 C-terminal domain and IL-15 N-terminal domain (L1 of FIG. 2), as well as between the IL-15 N-terminal domain and Fc domain (L2 of FIG. 2). The linker length was revealed to be critical for maintaining contact between the IL-15 and IL-15Rα sushi domain in the wild-type ligand-receptor complex. Finally, single- and double-point mutations were engineered to explore the range of IL-15 activity.

All variants were cloned and expressed in HEK293T cells. These variants were then tested for biochemical activity, thermal stability, and efficacy (Examples 2 and 3).

### Example 2: In Vitro Testing of IL-15/IL-15Rα-Fc Fusion Proteins

IL-15/IL-15Rα-Fc fusion proteins expressed in HEK293T cells were purified and analyzed for biochemical activity characterized in binding affinity to IL-2Rβ (CD122).

Surface plasmon resonance (SPR; Biacore system) was used to measure the dissociation constant (KD) between the IL-15/IL-15Rα-Fc fusion protein and IL-2Rβ. KD values represent a quantitative measurement of receptor affinity for the target ligand, where a lower KD value represents a higher affinity of the receptor (i.e., IL-2Rβ) for the target ligand (i.e., IL-15/IL-15Rα-Fc).

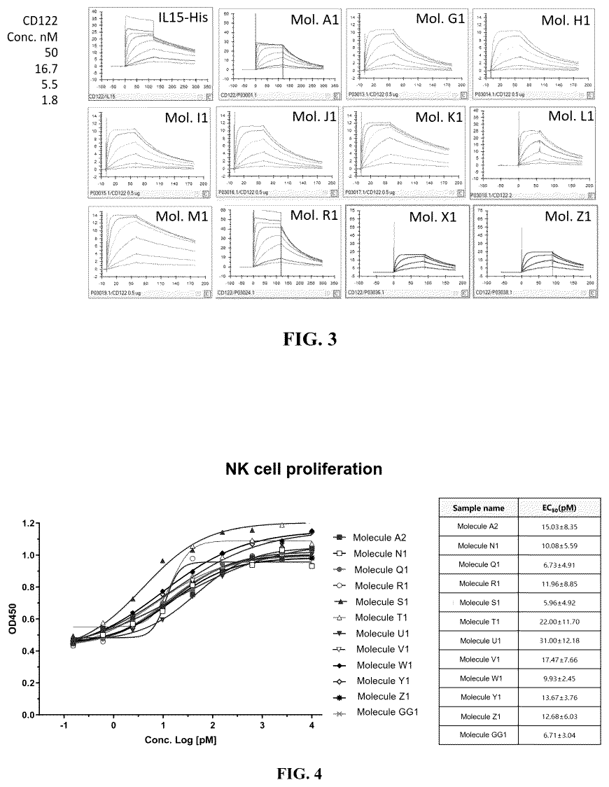

The results of the biochemical activity of IL-15/IL-15Rα-Fc fusion proteins are shown in FIG. 3 and Table 7. KD values ranged between 0.52 nM and 5.21 nM, indicating that IL-15/IL-15Rα-Fc fusion proteins produced a range of both increased and decreased affinity for IL-2Rβ.

TABLE 7

| | |
|---|---|
| Binding to IL-2Rβ | Thermal Stability |

| Molecule ID | KD | Tm 1: ° C. | Tm 2: ° C. | Tm 3: ° C. |
|---|---|---|---|---|
| Molecule A1 | 3.5 | 68.00 | 78.00 | 83.0 |
| Molecule B1 | 3.3 | 68.00 | 75.00 | 82.5 |
| Molecule C1 | 3.8 | 66.50 | 77.50 | 82.0 |
| Molecule D1 | 3.3 | 66.50 | 74.50 | 83.0 |
| Molecule E1 | 3 | 67.50 | 75.50 | 83.0 |
| Molecule F1 | 0.53 | 66.00 | N.O | 83.5 |
| Molecule G1 | 4.47 | 81.50 | | |
| Molecule H1 | 5.21 | 79.00 | | |
| Molecule I1 | 4.19 | 81.50 | | |
| Molecule J1 | 4.43 | 83.00 | | |
| Molecule K1 | 1.04 | 83.00 | | |
| Molecule L1 | 4.04 | 77.50 | | |
| Molecule M1 | 0.96 | 77.50 | | |
| Molecule N1 | 3.08 | 68 | 78 | n.r. |
| Molecule O1 | 3.15 | 59.5 | 78 | n.r. |
| Molecule P1 | 2.84 | 68 | 75.5 | 81.5 |
| Molecule Q1 | 2.65 | 68 | 78 | n.r. |
| Molecule R1 | 2.85 | 68 | 80 | n.r. |
| Molecule S1 | 0.6 | 68 | 79 | n.r. |
| Molecule T1 | 2.8 | 59.5 | 67.5 | 79 |
| Molecule U1 | 2.47 | 68 | 75 | 82 |
| Molecule V1 | 0.52 | 68 | 74.5 | 82 |
| Molecule AA1 | | 63.5 | n.r. | 82 |
| Molecule BB1 | 3.37 | 68.00 | 78.00 | n.r. |
| Molecule CC1 | 0.83 | 68.00 | 77.00 | n.r. |
| Molecule DD1 | 0.72 | 68.00 | 77.50 | n.r. |
| Molecule W1 | 3.42 | 68.00 | n.r. | 80.00 |
| Molecule EE1 | 3.3 | 59.50 | n.r. | 79.50 |
| Molecule FF1 | 3.03 | 68.50 | n.r. | 80.00 |
| Molecule X1 | 3.23 | 67.00 | n.r. | 80.00 |
| Molecule Y1 | 3.17 | 68.50 | n.r. | 80.00 |
| Molecule Z1 | 3.04 | 67.00 | n.r. | 80.00 |
| Molecule GG1 | 0.73 | 68.00 | 78.50 | n.r. |
| Molecule HH1 | 0.82 | 59.50 | 78.50 | n.r. |
| Molecule II1 | 0.73 | 68.50 | 79.00 | n.r. |
| Molecule JJ1 | 0.72 | 67.00 | 79.00 | n.r. |
| Molecule KK1 | 0.79 | 59.50 | 79.00 | n.r. |
| Molecule LL1 | 0.71 | 68.50 | 79.00 | n.r. |
| Molecule MM1 | 0.7 | 67.00 | 79.00 | n.r. |

The molecular attributes of IL-15/IL-15Rα-Fc fusion proteins were also assessed by evaluating thermal stability (Table 7) and dynamic light scattering (data not shown).

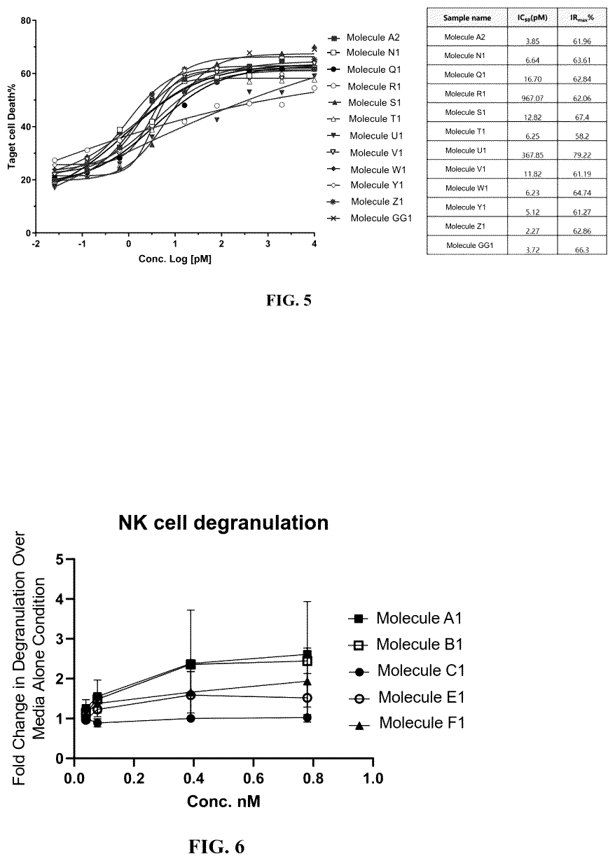

A subset of these IL-15/IL-15Rα-Fc fusion proteins were then selected for further functional testing in vitro. FIG. 4 shows the effects of IL-15/IL-15Rα-Fc fusion proteins on NK cell proliferation and cytotoxicity. NK92 cells are an interleukin-2 (IL-2) dependent natural killer (NK) cell line and were used to assess the effect of IL-15/IL-15Rα-Fc fusion proteins on NK cell proliferation and cytotoxicity. FIG. 4 shows that treatment of NK92 cells with increasing concentrations of IL-15/IL-15Rα-Fc fusion protein increased proliferation. FIG. 5 shows that treatment of NK92 cells with increasing concentrations of IL-15/IL-15Rα-Fc fusion protein increased target cell death.

IL-15/IL-15Rα-Fc fusion proteins were then assessed for their effect on NK cell degranulation. NK cells were treated with various concentrations of IL-15/IL-15Rα-Fc fusion proteins overnight. NK cell degranulation was analyzed with anti-CD107a antibody and flow cytometry. FIG. 6 shows that treatment of NK cells with increasing concentrations of IL-15/IL-15Rα-Fc fusion protein increased NK cell degranulation compared to untreated NK cells.

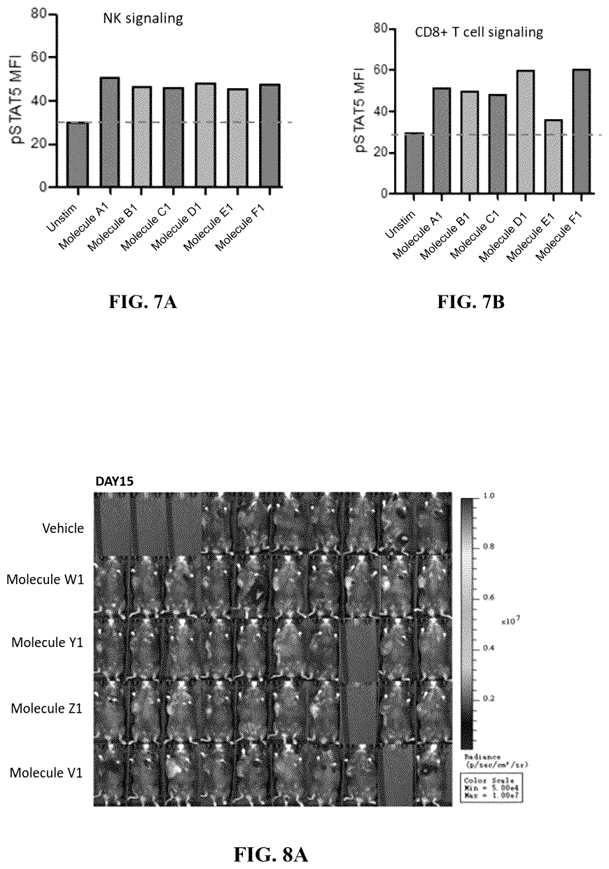

The effects of IL-15/IL-15Rα-Fc fusion proteins on NK and CD8+ T cell signaling was also assessed in vitro. FIG. 7A shows phosphorylation of STAT5 in primary NK cells treated with 0.5 nM of IL-15/IL-15Rα-Fc fusion proteins overnight. FIG. 7B shows phosphorylation of STAT5 in primary CD8+ T cells treated with 0.5 nM of IL-15/IL-15Rα-Fc fusion proteins overnight. STAT5 phosphorylation was analyzed with anti-phospho-STAT5 antibody and flow cytometry. FIG. 7A and FIG. 7B demonstrates that IL-15/IL-15Rα-Fc fusion proteins increase NK and CD8+ T cell activation.

Based on these in vitro testing results, the IL-15/IL-15Rα-Fc fusion proteins Molecule T1, Molecule W1, Molecule Y1, and Molecule Z1 were selected for testing in vivo (Example 3).

### Example 3: In Vivo Testing of IL-15/IL-15Rα-Fc Fusion Proteins

IL-15/IL-15Rα-Fc fusion proteins were further tested in vivo for their ability to inhibit tumor growth and metastasis in mice.

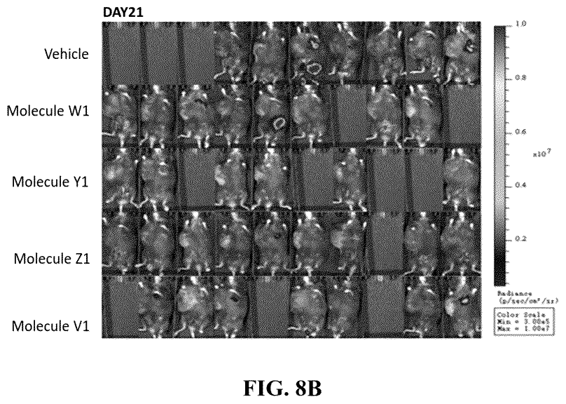

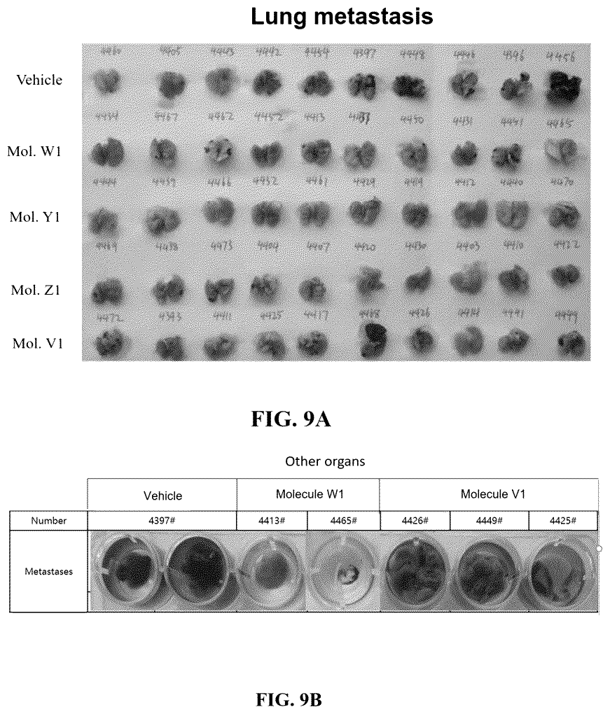

Tumor-bearing mice were administered vehicle (negative control), reference Molecule V1, Molecule W1, Molecule Y1, or Molecule Z1. Mice were sacrificed on Day 15 or Day 21 post-administration (FIG. 8A and FIG. 8B, respectively). Overall, treatment with the IL-15/IL-15Rα-Fc fusion proteins was well-tolerated. Of the four variants tested, Molecule W1, Molecule Y1, and Molecule Z1 demonstrated superior inhibition of tumor metastasis. Harvested lungs revealed varying degrees of tumor burden (black) depending on vehicle or variant administered (FIG. 9A). Other harvested organs demonstrated similar results to lungs, where Molecule Z1 exhibited superior reduction in tumor burden as compared to vehicle control and the standard reference Molecule V1 (FIG. 9B).

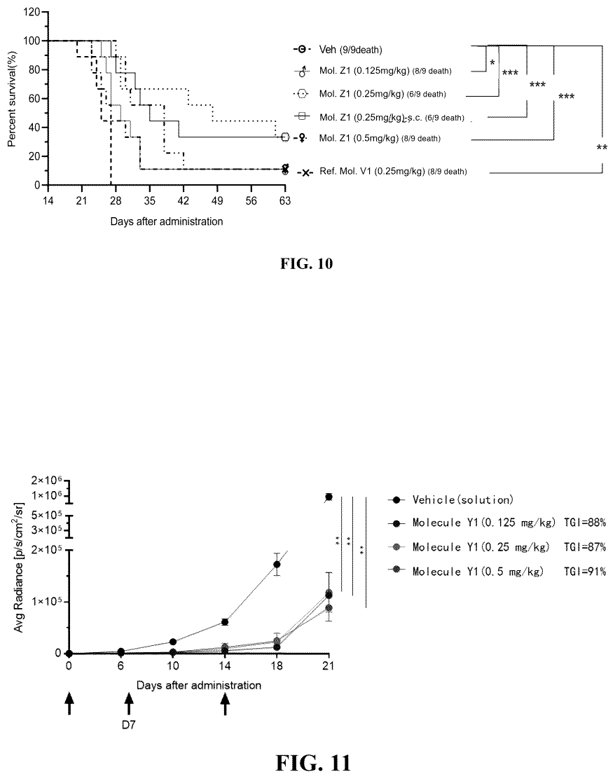

The IL-15/IL-15Rα-Fc fusion protein Molecule Z1 was further tested for its effects on survival in tumor-bearing mice. Molecule Z1 was administered to tumor-bearing mice either subcutaneously or intravenously at various doses. FIG. 10 shows that Molecule Z1 improved survival in tumor-bearing mice at all doses tested compared to vehicle control. Intravenous or subcutaneous administration of Molecule Z1 at a dose of 0.25 mg/kg demonstrated the greatest impact on survival compared to other doses examined.

Overall, these results demonstrate that treatment with IL-15/IL-15Rα-Fc fusion protein inhibits tumor growth and metastasis, and increases survival in a mouse model of cancer.

### Example 4: Animal Model Efficacy-Evaluation of the Efficacy of IL-15/IL-15Rα-Fc Fusion Protein on Mouse Melanoma Cells B16F10-Luc in a C57BL/6J Mouse Lung Metastasis Model

To evaluate the efficacy of IL-15/IL-15Rα-Fc fusion proteins in mouse models, murine melanoma cells B16F10-Luc transfected with Luciferase were selected with C57BL/6J mice to evaluate the effect of IL-15/IL-15Rα-Fc fusion proteins on the metastatic effects of murine melanoma cells B16F10-Luc in C57BL/6J mice in an anti-tumor cell metastasis model of lung metastasis via tail vein injection. A total of four groups were designed, and the experimental design, dosage, and mode of administration are shown in Table 8, and the changes in body weight of the loaded mice are shown in Table 9.

TABLE 8
Mice grouping and dosing regimen

| | | | | Frequency | Route of |
|---|---|---|---|---|---|
| | | | Dose | of adminis- | adminis- |
| Group | Test article | N | (mg/kg) | tration | tration |
| G1 | Vehicle (solution) | 10 | — | QW × 3 | i.p. |
| G2 | Molecule Y1 | 10 | 0.125 | QW × 3 | i.p. |
| G3 | Molecule Y1 | 10 | 0.25 | QW × 3 | i.p. |
| G4 | Molecule Y1 | 10 | 0.5 | QW × 3 | i.p. |

TABLE 9
Body weight of tumor-burdened mice

| | | Dose | Animal Number | | | Body weight (g) | | | | |
|---|---|---|---|---|---|---|---|---|---|---|
| Group | Test article | (mg/kg) | D 0 | D 21 | D 70 | D 0 | D 3 | D 5 | D 7 | D 21 |
| G1 | Vehicle | — | 10 | 9 | 0 | 19.2 ± 0.2 | 18.8 ± 0.2 | 18.5 ± 0.4 | 17.9 ± 0.3 | 19.4 ± 0.4 |
| | (solution) | | | | | | | | | |
| G2 | Molecule Y1 | 0.125 | 10 | 10 | 0 | 19.2 ± 0.1 | 19.5 ± 0.2** | 19.7 ± 0.2** | 19.0 ± 0.3* | 19.9 ± 0.2 |
| G3 | Molecule Y1 | 0.25 | 10 | 10 | 0 | 19.2 ± 0.2 | 18.6 ± 0.1 | 17.8 ± 0.1* | 17.0 ± 0.3* | 20.0 ± 0.2 |
| G4 | Molecule Y1 | 0.5 | 10 | 10 | 1 | 19.2 ± 0.1 | 18.5 ± 0.2 | 17.6 ± 0.1** | 17.2 ± 0.1 | 20.8 ± 0.3** |

*P < 0.05 V.S.G1,
**P < 0.01 V.S.G1

Evaluation of the efficacy of IL-15/IL-15Rα-Fc fusion proteins on mouse melanoma cells B16F10-Luc in a C57BL/6J mouse lung metastasis model-Radiance values of lung metastatic tumors from tumor-burdened mice are detailed in Table 10 and FIG. 11.

TABLE 10
Radiance values of lung metastatic
tumors from tumor-burdened mice

| | | | Avg Radiance(p/s/cm²/ | TGI |
|---|---|---|---|---|
| Group | Test article | Dose(mg/kg) | sr) − D 21 | % |
| G1 | Vehicle (solution) | — | 9.48E+05 ± 2.27E+05 | — |
| G2 | Molecule Y1 | 0.125 | 1.13E+05 ± 4.43E+04 | 88 |
| G3 | Molecule Y1 | 0.25 | 1.19E+05 ± 3.85E+04 | 87 |
| G4 | Molecule Y1 | 0.5 | 8.86E+04 ± 2.57E+04 | 91 |

As shown in Table 10 and FIG. 11, at day 21, the Tumor Growth Inhibition value (TGI%) of B16F10-Luc lung metastatic tumors in the monotherapy group of Molecule Y1 at the doses of 0.125, 0.25, and 0.5 mg/kg were 88%, 87%, and 91%, respectively, which were all statistically highly significant compared with that of the control group (P<0.01). It can be seen that molecule Y1 has good efficacy and safety.

In conclusion, these results suggest that treatment of mouse tumor models with IL-15/IL-15Rα-Fc fusion proteins inhibits tumor growth and metastasis, and further improves survival in mouse tumor models.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A polypeptide comprising from N-terminus to C-terminus:
(a) an IL-15 receptor alpha sushi domain;
(b) an IL-15 C-terminal domain;
(c) an IL-15 N-terminal domain; and
(d) an Fc domain.

2. The polypeptide according to claim 1, wherein the IL-15 receptor alpha sushi domain comprises an amino acid sequence with at least about 85%, at least about 90%, at least about 95%, at least about 96%, at least about 97%, at least about 98%, or at least about 99% sequence identity to any one of SEQ ID NOs: 8-11, or further preferably, wherein the IL-15 receptor alpha sushi domain comprises an amino acid sequence of any one of SEQ ID NOs: 8-11.

3. The polypeptide according to claim 1, wherein the IL-15 C-terminal domain comprises an amino acid sequence with at least about 85%, at least about 90%, at least about 95%, at least about 96%, at least about 97%, or at least about 98% sequence identity to SEQ ID NO: 3 or SEQ ID NO: 4, or further preferably, wherein the IL-15 C-terminal domain comprises an amino acid sequence of SEQ ID NO: 3 or SEQ ID NO: 4.

4. The polypeptide according to claim 1, wherein the IL-15 N-terminal domain comprises an amino acid sequence with at least about 85%, at least about 90%, at least about 95%, at least about 96%, at least about 97%, or at least about 98% sequence identity to SEQ ID NO: 5 or SEQ ID NO: 6, or further preferably, wherein the IL-15 N-terminal domain comprises an amino acid sequence of SEQ ID NO: 5 or SEQ ID NO: 6, or further preferably, wherein the IL-15 N-terminal domain comprises an N72D mutation.

5. The polypeptide according to claim 1, wherein the Fc domain is an IgG1 Fc domain comprising an amino acid sequence with at least about 85%, at least about 90%, at least about 95%, at least about 96%, at least about 97%, at least about 98%, or at least about 99% sequence identity to SEQ ID NO: 12, or further preferably, wherein the Fc domain is an IgG1 Fc domain comprising an amino acid sequence of SEQ ID NO: 12 or SEQ ID NO: 14; or
the Fc domain is an IgG4 Fc domain comprising an amino acid sequence with at least about 85%, at least about 90%, at least about 95%, at least about 96%, at least about 97%, at least about 98%, or at least about 99% sequence identity to SEQ ID NO: 17, or further preferably, wherein the Fc domain is an IgG4 Fc domain comprising an amino acid sequence of SEQ ID NO: 17.

6. (canceled)

7. The polypeptide according to claim 5, wherein the IgG1 or IgG4 Fc domain comprises at least one amino acid substitution, or further preferably, wherein the at least one amino acid substitution is N297A, L234A, L235A, and/or P329G, or further preferably, wherein the at least one amino acid substitution is M252Y, S254T, T256E, or a combination thereof, or further preferably, wherein the at least one amino acid substitution is L234A, L235A, or a combination thereof.

8. The polypeptide according to claim 1, wherein the polypeptide comprises one or more linkers;
preferably, the polypeptide comprises a linker between the IL-15 C-terminal domain and the IL-15 N-terminal domain, or/and, wherein the polypeptide comprises a linker between the IL-15 N-terminal domain and the Fc domain.

9. (canceled)

10. The polypeptide according to claim 8, wherein the one or more linkers is 2, 3, 4, 5, 6, 7, 8, 9, or 10 amino acids in length, or further preferably, wherein the one or more linkers is 5 amino acids in length, or further preferably, wherein the one or more linkers comprise an amino acid sequence selected from the group consisting of: GG, GS, GGS, GGGS, and GGGGS.

11. The polypeptide according to claim 1, wherein the polypeptide comprises an amino acid sequence with at least about 85%, at least about 90%, at least about 95%, at least about 96%, at least about 97%, at least about 98%, or at least about 99% sequence identity to any one of SEQ ID NOs: 20-30; or
the polypeptide comprises an amino acid sequence of any one of SEQ ID NOs: 20-30, or further preferably, wherein the polypeptide consists of an amino acid sequence of SEQ ID NOS: 24, 27, 28 or 30.

12. (canceled)

13. The polypeptide according to claim 1, wherein the polypeptide comprises an amino acid sequence of any one of SEQ ID NOs: 20-30 with one or more mutations, or further preferably, wherein the polypeptide comprises an amino acid sequence of SEQ ID NO: 24 with one or more mutations in IL-15 N domain selected from the group consisting of: V3A, V3L, N4A, N4D, N4Q, S7D, S7V, D8T, D8A, K11A, D30N, D61A, D61S, D61N, D61E, E64Q, N65D, I68A, I68V, L69A, L69V, N72E and N72A.

14. The polypeptide according to claim 1, wherein the polypeptide comprises an amino acid sequence of SEQ ID NO: 28 with one or more mutations selected from the group consisting of: N189Q and G190A; or
the polypeptide comprises an amino acid sequence of SEQ ID NO: 30 with one or more mutations selected from the group consisting of: N189Q and G190A.

15. (canceled)

16. The polypeptide according to claim 1, wherein the polypeptide consists of an amino acid sequence of any one of SEQ ID NOs: 20-30.

17. The polypeptide according to claim 1, wherein the polypeptide binds to IL-2 receptor beta and/or IL-2 receptor gamma, further preferably, wherein the polypeptide binds to IL-2 receptor beta with an affinity of about 0.1 nM to about 25 nM, about 0.5 nM to about 10 nM, or about 1 nM to about 5 nM.

18. A polynucleotide encoding the polypeptide according to claim 1.

19. An expression vector comprising the polynucleotide of claim 18.

20. A host cell comprising the polynucleotide of claim 18.

21. A pharmaceutical composition comprising the polypeptide of claim 1, and at least one pharmaceutically acceptable carrier or excipient.

22. A method of treating cancer, the method comprising administering to a subject in need thereof an effective amount of the polypeptide of claim 1.

23. The method of claim 22, wherein the cancer is a solid tumor.

24. The method of claim 23, wherein the cancer is metastatic.


SEQUENCE LISTING (31 sequences)

SEQ ID NO: 1 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 267 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 3 - protein, 36 aa, synthetic construct
NVTESGCKECEELEEKNIKEFLQSFVHIVQMFINTS

SEQ ID NO: 4 - protein, 34 aa, synthetic construct
TESGCKECEELEEKNIKEFLQSFVHIVQMFINTS

SEQ ID NO: 5 - protein, 78 aa, synthetic construct
NWVNVISDLKKIEDLIQSMHIDATLYTESDVHPSCKVTAMKCFLLELQVISLESGDASIHDTVENLIILANNSLSSNG

SEQ ID NO: 6 - protein, 80 aa, synthetic construct
NWVNVISDLKKIEDLIQSMHIDATLYTESDVHPSCKVTAMKCFLLELQVISLESGDASIHDTVENLIILANNSLSSNGNV

SEQ ID NO: 7 - protein, 80 aa, synthetic construct
NWVNVISDLKKIEDLIQSMHIDATLYTESDVHPSCKVTAMKCFLLELQVISLESGDASIHDTVENLIILANDSLSSNGNV

SEQ ID NO: 8 - protein, 78 aa, synthetic construct
ITCPPPMSVEHADIWVKSYSLYSRERYICNSGFKRKAGTSSLTECVLNKATNVAHWTTPSLKCIRDPALVHQRPAPPS

SEQ ID NO: 9 - protein, 73 aa, synthetic construct
ITCPPPMSVEHADIWVKSYSLYSRERYICNSGFKRKAGTSSLTECVLNKATNVAHWTTPSLKCIRDPALVHQR

SEQ ID NO: 10 - protein, 68 aa, synthetic construct
ITCPPPMSVEHADIWVKSYSLYSRERYICNSGFKRKAGTSSLTECVLNKATNVAHWTTPSLKCIRDPA

SEQ ID NO: 11 - protein, 65 aa, synthetic construct
ITCPPPMSVEHADIWVKSYSLYSRERYICNSGFKRKAGTSSLTECVLNKATNVAHWTTPSLKCIR

SEQ ID NO: 12 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 232 aa, synthetic construct
EPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 11 aa, synthetic construct
GSGGGGSGGGS

SEQ ID NO: 19 - protein, 5 aa, synthetic construct
GGGGS

SEQ ID NO: 20 - protein, 434 aa, synthetic construct
ITCPPPMSVEHADIWVKSYSLYSRERYICNSGFKRKAGTSSLTECVLNKATNVAHWTTPSLKCIRDPALVHQRPAPPSNVTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 434 aa, synthetic construct
ITCPPPMSVEHADIWVKSYSLYSRERYICNSGFKRKAGTSSLTECVLNKATNVAHWTTPSLKCIRDPALVHQRPAPPSNVTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGGGGGSEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 424 aa, synthetic construct
ITCPPPMSVEHADIWVKSYSLYSRERYICNSGFKRKAGTSSLTECVLNKATNVAHWTTPSLKCIRDPANVTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 434 aa, synthetic construct
ITCPPPMSVEHADIWVKSYSLYSRERYICNSGFKRKAGTSSLTECVLNKATNVAHWTTPSLKCIRDPALVHQRPAPPSTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGNVGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 429 aa, synthetic construct
ITCPPPMSVEHADIWVKSYSLYSRERYICNSGFKRKAGTSSLTECVLNKATNVAHWTTPSLKCIRDPALVHQRTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGNVGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 429 aa, synthetic construct
ITCPPPMSVEHADIWVKSYSLYSRERYICNSGFKRKAGTSSLTECVLNKATNVAHWTTPSLKCIRDPALVHQRTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANDSLSSNGNVGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 429 aa, synthetic construct
ITCPPPMSVEHADIWVKSYSLYSRERYICNSGFKRKAGTSSLTECVLNKATNVAHWTTPSLKCIRDPALVHQRTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGNVGGGGSEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 429 aa, synthetic construct
ITCPPPMSVEHADIWVKSYSLYSRERYICNSGFKRKAGTSSLTECVLNKATNVAHWTTPSLKCIRDPALVHQRTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGNVGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 429 aa, synthetic construct
ITCPPPMSVEHADIWVKSYSLYSRERYICNSGFKRKAGTSSLTECVLNKATNVAHWTTPSLKCIRDPALVHQRTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGNVGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 424 aa, synthetic construct
ITCPPPMSVEHADIWVKSYSLYSRERYICNSGFKRKAGTSSLTECVLNKATNVAHWTTPSLKCIRDPALVHQRTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGNVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 424 aa, synthetic construct
ITCPPPMSVEHADIWVKSYSLYSRERYICNSGFKRKAGTSSLTECVLNKATNVAHWTTPSLKCIRDPALVHQRTESGCKECEELEEKNIKEFLQSFVHIVQMFINTSGGGGSNWVNVISDLKKIEDLIQSMHIDATLYTESDVHPSCKVTAMKCFLLELQVISLESGDASIHDTVENLIILANNSLSSNGNVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 448 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSGGNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK